(19) United States
(12) Patent Application Publication
(10) Pub. No.: US 2015/0100242 A1
(43) Pub. Date: Apr. 9, 2015

(54) METHOD, KIT AND ARRAY FOR BIOMARKER VALIDATION AND CLINICAL USE

(71) Applicant: QIAGEN SCIENCES LLC, Germantown, MD (US)

(72) Inventors: Xiao Zeng, Frederick, MD (US); Song Tian, Germantown, MD (US); Jiaye Yu, Durham, NC (US); John Dicarlo, Bethesda, MD (US); George J. Quellhorst, JR., Rockville, MD (US); Vikram Devgan, Frederick, MD (US)

(21) Appl. No.: 14/384,913

(22) PCT Filed: Mar. 15, 2013
(86) PCT No.: PCT/US13/32118

§ 371 Date: Sep. 12, 2014

Related U.S. Application Data
(60) Provisional application No. 61/611,173, filed on Mar. 15, 2012.

(51) Int. Cl. C12Q 1/68; G06F 19/10

(52) U.S. Cl. CPC C12Q 1/686; G06F 19/10
USPC 702/19; 506/17; 506/9

(57) ABSTRACT

The methods provided focus on a quantitative molecular assay tools that systematically measure a set of pre-selected targets, with proper controls in a biological sample for identification of biomarkers or novel targets for a disease status. This allows for systematically maximizing the power of multivariate feature selection tools on the analysis of high-throughput screening data (such as microarray) and use of the well selected target to generate a qPCR array with tissue specific controls and qPCR controls to serve the needs of biomarker study.

### BACKGROUND OF THE INVENTION

1. Field of the Invention

The methods provided focus on a quantitative molecular assay tools that systematically measure a set of preselected targets, with proper controls in a biological sample for identification of biomarkers or novel targets for a disease status. This allows for systematically maximizing the power of multivariate feature selection tools on the analysis of high-throughput screening data (such as microarray) and use of the well selected targets to generate a quantitative real-time (qPCR) array with tissue specific controls and qPCR controls to serve the needs of biomarker study.

2. Background of the Invention

Challenges in clinical disease classification and drug response estimation are increasing. Traditional concepts for disease diagnoses and drug developments have reached a bottleneck. Some novel personalised medicine concepts, however, have been well accepted. Biomarkers, especially molecular biomarkers, are taking a leading role in this new orientation. Unfortunately, substantial work is wasted in biomarker research due to inefficient exploration of data from precious clinical samples. The challenge is not more high-throughput screening, but instead exploring valuable targets from the known information and converting the assay in a more practical way for biomarker(s) identification. Assays, such as qPCR arrays, are required to be performed at an industry-standardized level in order to protect the assays' sensitivity, accuracy and consistency.

There is therefore a need for a systematic solution for the development of biomarkers, especially for the gene expression signature. Most biomarkers are based on microarray analysis, but many do not include further platform conversion. Even those that include second platform validation still do not involve further feature selection and classification based on the new platform used.

Additionally, microarray-based assays have some inherent drawbacks. They are sensitive to sample quality, which often presents challenges for clinical samples. They also require increased sample preparation time and complicated data analysis procedures.

Platforms such, as qPCR, which are utilized in clinical diagnosis practice, are limited to individual cases (diseases). Additionally, those using platforms such as qPCR do not provide a systematic method for biomarker selection and validation. Moreover, the majority of those using such platforms do not use a genome-wide feature selection process, thus limiting their potential to select the best marker from genome wide targets.

### SUMMARY OF THE INVENTION

In embodiments, methods of preparing a biomarker quantitative real-time polymerase chain reaction (qPCR) array are provided. Suitably, the methods comprise selecting one or more high-throughput feature expression data sets, normalizing the feature expression data sets, analyzing the data sets by one or more mathematical models to yield final candidate features, and generating the biomarker qPCR array comprising the final candidate features.

Suitably, the one or more high-throughput feature expression data sets are selected based on one or more of clinical utility, research interest, drug response, species and quality. In embodiments, the analyzing comprises analysis with one or more mathematical models selected from Random Forest (RF) modeling. Support Vector Machine (SVM) modeling and Nearest Shrunken Centroid (NSC) modeling. In further embodiments, the analyzing comprises combining discriminative features from one or more of the mathematical models based on a desired classification implied by the data sets.

Suitably, the analysing further comprises literature mining to yield the final candidate features.

In additional embodiments, the methods further comprise selecting one or more control data sets for inclusion of control features in the biomarker qPCR array.

Also provided are qPCR arrays prepared by the methods described herein, suitably where each defined location in the array corresponds to a biological target.

In embodiments, die qPCR array is for analysis of messenger RNA (mRNA), or the qPCR array is for analysis of micro RNA (miRNA), or the qPCR array is for analysis of long non-coding RNA (lncRNA).

In suitable embodiments, the arrays comprise five or more control features selected from, but not limited to, ACTB, B2M, GUSB, HPRT1, RPL13A, S100A6, TFRC YWHAZ, CFL1, RPS13, TMED10, UBB, ATP5B, GAPDH, HMBS, HSPCB, RPLPO, SDHA, UBC, PPIA, FLOT2, TMB1M6, TBT1, MRPL19 and RPLP0.

In further embodiments, methods of assigning a single probability score to one or more biomarkers are provided, Suitably, the methods comprise collecting a sample set, extracting nucleic acid molecules from each sample of the sample set, interrogating each nucleic acid molecule with the qPCR array described herein and evaluating the discrimination power of one or more independent features, generating a combined feature by normalizing the one or more independent features and evaluating the feature's discrimination power, and assigning a single probability score to the combined features.

In suitable embodiments, the interrogating comprises evaluating 2 to 40 independent features, for example, 2 to 8 independent features, 8 to 16 independent features, 16 to 24 independent features, 24 to 32 independent features, 32 to 40 independent features, or 20 independent features.

### BRIEF DESCRIPTION OF THE DRAWINGS

FIGS. 1A-1B show Biomarker qPCR Array format examples in accordance with embodiments described herein. Txxx: assay target, HKx: reference genes, GDC: genomic DNA contamination control. RTC: reverse transcription efficiency control. PPC: qPCR performance control (A) 384-well format, (B) 96-well format.

FIG. 2 shows an example of a development roadmap for preparing a biomarker qPCR array as described herein.

FIG. 3 shows a biomarker qPCR array development process as described herein.

FIG. 4 shows a workflow from sample to biomarker signature panel using the biomarker qPCR array system as described herein.

FIGS. 5A-5D show the development of a thyroid malignancy qPCR array, as described herein.

FIG. 6 shows the results of a thyroid malignancy signature.

FIG. 7 shows an unsupervised hierarchical clustering of all relative gene expression levels in all samples roughly segregates the samples into the pre-defined TB-infected and control groups. In this heat map representation, the left y-axis displays the clustering of the original known sample types. TB-infected samples (TB) and healthy control sampled (C) clusters are indicated. The right y-axis lists the sample ID. The top x-axis displays the clustering of the genes (not labeled for simplicity and clarity). One TB-infected sample (TC0185) and two healthy control samples (TC3387 and TBC9588) seem to misclassify in this analysis.

FIG. 8 shows a Principle Component Analysis also roughly segregates the samples into the pre-defined TB-infected and control groups. In the analysis result, most of the TB-infected samples and the healthy control cluster together as two separate groups, with the exception of two misclassified samples shared by the cluster analysis (FIG. 7): TC0185 (TB-infected) and TBC95888 (healthy control).

FIG. 9 shows a random forest algorithm identifies the top ranked genes by importance. The importance of each gene (y-axis) based on its classification power was calculated with the random forest model as described, and plotted versus the gene symbol for the top ranked 16 genes (x-axis). The higher the y-axis value is, the greater the importance is. Genes increase in importance from left to right.

FIG. 10 shows an evaluation of the 16-gene signature panel classification model reveals that it segregates the samples into the pre-defined groups well, but still misclassifies two samples. The plot displays the probability (x-axis) that each sample (ID listed on the y-axis) classifies into the TB-infected group. TB-infected samples (positive), and healthy control samples (negative) are shown. Most samples correctly classify into the groups, except for the same two samples misclassified by the original PCA (TB-infected TC0185 and healthy control TBC95883). Three TB-infected samples (TC2615, Helios_TB07, and Helios_TB02) also seem to have “marginal calls” in that they do not have a 100% probability of being called TB-infected.

FIG. 11 shows an unsupervised hierarchical clustering using the 16-gene signature panel better discriminates the expression pattern of the known groups of samples, but might also be defining a new group or sub-group. The heat map representation is organized in the same fashion as FIG, 8. Samples that misclassify (TB-infected TC0185 and healthy control TBC95888) or have “marginal calls” in other analyses (TB-infected samples TC2615, Helios_TB07, and Helios₁₃ TB02) seem to cluster into a third group or sub-group.

### DETAILED DESCRIPTION OF PREFERRED EMBODIMENTS

It should be appreciated that the particular implementations shown and described herein are examples and are not intended to otherwise limit the scope of the application in any way.

The published patents, patent applications, websites, company names and scientific literature referred to herein are hereby incorporated by reference in their entireties to the same extent as if each was specifically and individually indicated to he incorporated by reference. Any conflict between any reference cited herein and the specific teachings of this specification shall be resolved in favor of the latter. Likewise, any conflict between an art—understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein, to mean approximately, in the region of roughly, or around. When the term “about” is used in conjunction with a numerical range. It modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of ordinary skill in the art.

Since microarray technology and molecular profiling became available for clinical specimens, thousands of disease-related biomarkers or disease signatures have been reported in the literature. However, the data analyses and interpretation of these signatures are not standardized. Consequently, only a handful of these “biomarkers” have been further validated and used in clinical practice, which is far from the expectation of the “power” of microarray technology that was initially portrayed to the general public. Part of the reason for this reality is that the end game for academic researchers is to share the difference, or the differentially expressed genes in diseased versus normal tissues because their primary goal is to “discover” and publish. But for clinicians, they already “expected” to see some kind of difference between diseased and normal tissues. What clinicians need is a cut-off score(s) to assign their patients into different groups and to make their treatment decisions accordingly in order to utilize the “discovery” in practice. Like the Valley of Death in drug development, which is the time frame between lead compound optimization and first-in-human clinical trials, there is a Valley of Death in biomarker development, which is defined as the time frame between “lead signature gene panel” optimisation and the first, in-human clinical trial.

The methods provided herein shorten the time frame that any given “feature signature panel” or “gene signature panel” stays in this Valley of Death. A major proposal provided is that the feature signature discovered on a microarray is converted onto a qPCR array, which fits the normal workflow in most of clinical labs. Although the concept of converting a microarray assay into a qPCR assay has been demonstrated in the literature, massive conversion of an assay panel, especially with the requirement that the PCR assay panel retain the classification power equal to, or better than the original microarray assays, has not been demonstrated. Also provided herein is a set of classification algorithms that guide the qPCR array users to validate the signature panel and eventually lead to a biomarker that fits the practical clinical need by giving the final readout as a score rather than a “profile” of up-and-down features or genes. Molecular detection methods such as real-time Polymerase Chain Reaction (PCR) are widely used in clinical molecular diagnosis. Even though clinical researchers understand the need for controls to monitor the input difference between samples so that they can be compared equally, they may not be aware that most of the controls that they use, which are found in most publications, lack sample-type specificity. Choosing the wrong controls is one of the reasons for the failure to validate some of the “biomarkers” published in the literature. Critical controls to monitor assay quality itself are also often neglected in the published literature, without which the systemic variation of the assay cannot be corrected for before the data are used for comparison. Provided herein is a description of how to choose the correct controls for qPCR arrays.

Multivariate biomarkers discovered on microarray platforms with tens of thousands of features are to be validated on a more practical assay platform, such as the qPCR platform,, in order to be accepted and practiced In clinical settings. Unfortunately, almost all “biomarkers” get stuck, at the discovery phase and never have a chance to see their true practical use in the clinic. What is worse is that when some of the discovered biomarkers are tested, they are found to be “unstable” or outright “fail” in clinical testing. The main reason for this “lack of confidence” sentiment is lack of the ability of high level data analysis and assay standardization and optimization in the initial as well as the follow-up studies.

Provided herein is a systematic method to 1) select multivariate features from published microarray datasets; 2) generate PCR arrays (e.g., quantitative real-time PCR (qPCR) arrays) with optimized assay design and proper controls; and 3) provide a companion algorithm that will finalize biomarker panels and generate a probability score for any clinical phenotype (disease type) under study.

The kit components described herein include an array of pre-dispensed PCR primers that are dried down on a qPCR plate. Each defined location within the array corresponds to a biological target (a gene or any nucleic acid molecule). Detection can be via qPCR using an appropriate reaction mixture and biological and pathological samples (such as cDNA reverse-transcribed from total RNA).

Also provided herein is a system that also includes a very unique control panel. A key issue in biomarker identification is the control. The expression of any given gene can be affected by tissue type, disease status and sample collection and storage conditions. Even some common housekeeping genes can be altered by disease conditions. Using a panel of well-selected normalization controls (reference genes), which better control the tissue sample amount used in each assay correctly, allows for an accurate comparison of the expression of certain genes is provided herein. The control panel also includes assay quality controls in order to help identify any condition that affects the evaluation of biomarker targets (for example the genomic DNA contamination in cDNA detection).

Also provided is a system that also includes a biomarker identification solution to allow for customer analysis of their data. The identification solution calculates the control genes' expression and provides further evaluation for the controls' performance in a real sample test. Finally, the identification solution helps users to select the best control genes for study. It also provides a ranking system that can rank the targets based on their importance when using them as biomarkers (for example the importance on disease status classification). It also provides a signature generation solution that provides the user with a panel of genes that can be used in a classification model as biomarkers.

A data set from high-throughput technology as well as the text mining gene list are used for final feature selection in thyroid malignancy identification. Several feature selection methods (such as Random forest and support vector machine) are used to rank the targets. With the selected gene, a 384-well qPCR array (including 10 selected specific thyroid nodule housekeeping genes and 3 qPCR assay controls) is used to study a set of 49 benign and malignant thyroid samples for the signature panel development. Five reference genes are further selected based on analysis. Using a random forest classification model, a fine toned classification classification signature (7 target genes and 5 controls) is developed. Besides the training set, the methods also work well on a test set that totally different from the training set. It can reach 91.7% accuracy, 87.5% sensitivity and 100% specificity, 100% PPV and 80% NPV. It also shows its power in a mixed sample test, which can identify a tumor sample that only contained 25% real malignant sample and mixed with 75% benign sample. These results suggest that the biomarker PCR array system described herein is an efficient tool for biomarker development.

The methods provided focus on a quantitative molecular assay tool that systematically measures a set of pre-selected targets, with proper controls in a biological sample for identification of biomarkers or novel targets for a disease status. This allows for systematically maximizing the power of multivariate feature selection tools on the analysis of high-throughput screening data (such as microarray) and use of the wed selected target to generate a qPCR array with tissue specific controls and qPCR controls to serve the needs of biomarker study.

Also provided are methods to select candidates' targets based on high-throughput screening data analysis and literature mining.

Public high-througbput analysis data sets are analyzed biologically, clinically and statistically for study topic and research subject, as well as data quality and sample grouping.

High-throughput analysis data set(s) with defined research topic(s) and good quality are processed to a standard that can be combined/compared and input into a bioinformatics model system(s).

Processed high-throughput analysis data are analysed and ranked with well-established statistical feature selection model system(s), such as Random forest, support vector machine, nearest shrunken centroid and bayesian factor regression modeling.

Research topics include disease classification, treatment response prediction, or pathway activation/inhibition. The research topics are used to mine the literature through publication databases in order to select the most important targets that studies have suggested play an important role in the defined topics as a marker. All the targets of interest are ranked based on their biomarker related importance.

Selected targets are combined by putting separate lists together or by re-ranking with the combination of all the different rankings. A final list (for example a 96-well or 384-well, depending on format) is generated by putting all of the most important gene targets together.

Provided herein is a system which includes an array of pre-dispensed and dried PCR primers, each at a defined location within the array that focuses on well analyzed and selected biological targets (a gene or any nucleic acid molecule).

Detection can be via qPCR using an appropriate reaction mixture and biological and pathological samples (such as cDNA reverse-transcribed from total RNA).

The assay for selected targets is designed and tested for its sensitivity, specificity and efficiency with an industry standard. The detection assay is specific, correlates well with input change and is sensitive enough for low expression detection.

The final list suitably includes those high-ranking targets with assays that fit the quality control standard.

Also provided is a system which includes a control panel:

A panel (5-20) of normalization controls (reference genes), which better controls the tissue sample amount used in each assay, to provide an accurate comparison of the expression of certain genes. The selected research topics are used to study the reference genes that can better represent sample input. A selected number of samples (tissue, cells or purified nucleic acids) that represent the selected topics are used to evaluate their reference stability and variation in detection with a defined detection method such as quantitative real-time PCR. The reference targets tested include, but not are limited to, ACTB, B2M, GUSB, HPRT1, RPL13A, S100A6, TFRC, YWHAZ. CFL1, RPS13, TMED10, UBB, ATP5B, GAPDH HMBS, HSPCB. RPLPO, SDHA, UBC, PP1A, FLOT2, TMB1M6, TBT1, MRPL19 and RPLP0. The reference genes can also be selected based on publications if the reference genes have been well studied for the selected research topic.

In an exemplary embodiment, a thyroid nodule malignancy classification gene panel as described herein comprises Targets genes: NPC2, S100A11, SDC4, CD53 MET, GCSH and CHI3L1, and Reference genes TBP, RPL13A, RPS13, HSP90AB1 and YWHAZ.

The control panel also includes, assay quality control to help identify any condition that affects the evaluation of biomarker targets (for example the genomic DNA contamination in cDNA detection). The included controls include GDC (genomic DNA contamination control), RTC (reverse transcription efficiency control) and PPC (qPCR performance control) or others that are valuable for assay quality control

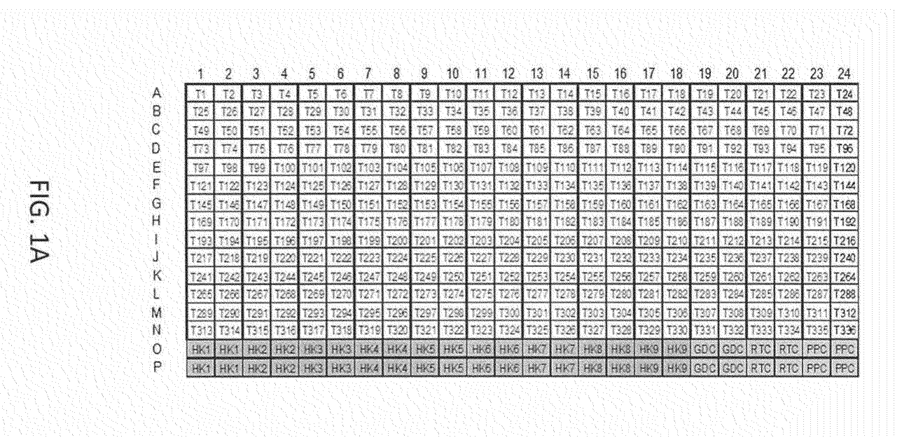

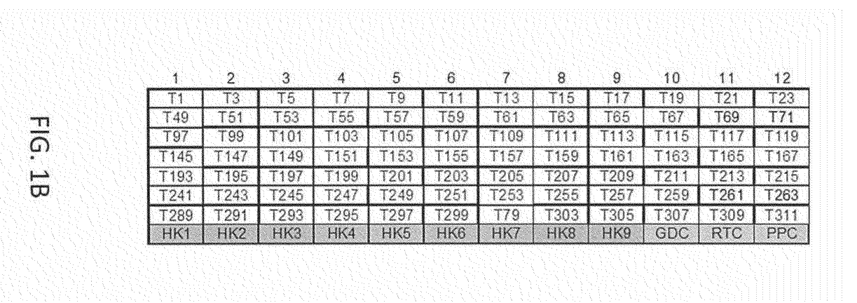

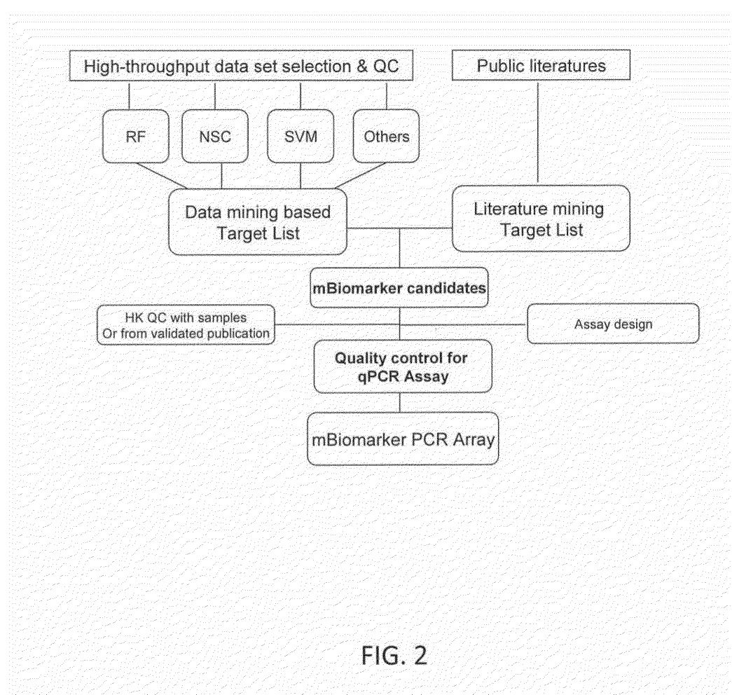

All of the controls are rearranged and finalized in a proper format (such as 384-well PCR plate or 96-well format) with the necessary assay material (such as qPCR primers) dispensed in the assigned location. See FIGS. 1A end 1B.

Also provided is a system which also includes a biomarker identification data analysis platform to allow for customer analysis of their data.

The systems disclosed herein provide QC analysis to help customers evaluate assay quality, the sample quality and potential outliers.

The systems disclosed herein calculate gene expression stability of the reference genes provided in the array system. The systems disclosed herein provide recommendations for the best reference genes to use for biomarker data analysis.

The systems disclosed herein provide a ranking system (such as Random forest based feature selection and ranking system) that can rank the targets based on their importance when using them as biomarkers (for example the importance on disease stains classification).

The systems disclosed herein provide a signature generation solution that provides users with a panel of genes that can be used in a classification model as biomarkers. A classification model is used with default settings to perform the analysis online. A customized analysis is also available as a part of a service. Suitable models include:
Random forest (RF) (R package randomForest),
nearest shrunken centroids (NSC),
bayesian factor regression modeling (BFRM),
support vector machine (SVM) (SVM implementation in the libSVM software library,
Bayesian factor regression modeling (BFRM) (from West group),
Hierarchical clustering, and
Heatmap analysis.

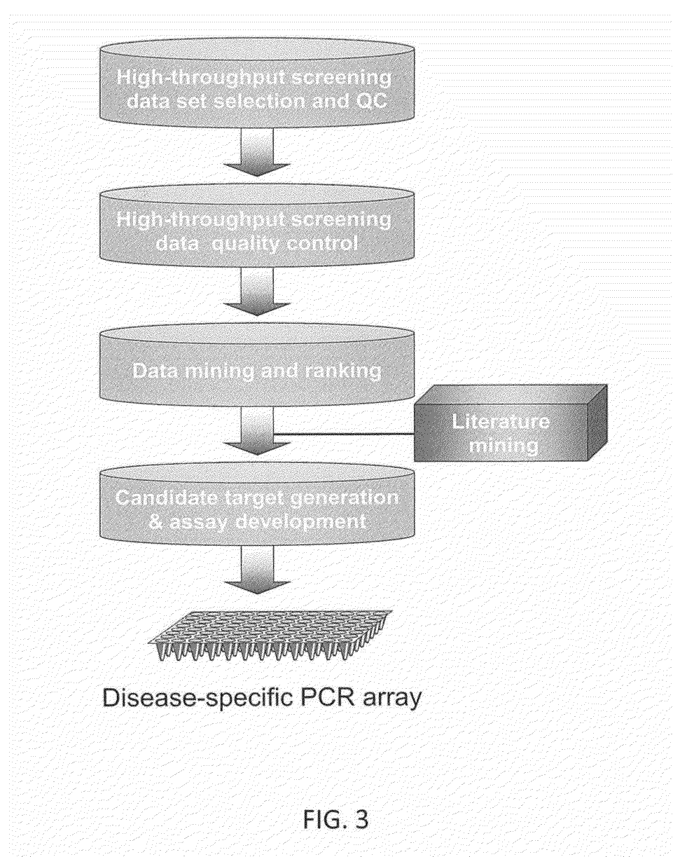

As shown In FIG. 3, in embodiments, high-throughput gene expression, data sets are selected based on research interest study objective, species and quality [minimum sample numbers, well-defined sampling conditions, availability of annotation, and uniformity of experimental data (signal intensify, outliers etc.)].

Selected data sets are normalized and then analyzed by multiple mathematical models including Random forest (RF), support vector machine (SVM) and nearest shrunken centroid (NSC). Top-ranked targets from all statistical analyzes and literature mining are combined to produce the final candidate gene list.

Quantitative real time (qPCR) assays for all candidate genes are designed and tested for technical sensitivity, specificity, and dynamic range. Tissue-specific normalization control assays and performance controls are added to complete the final disease-specific qPCR array.

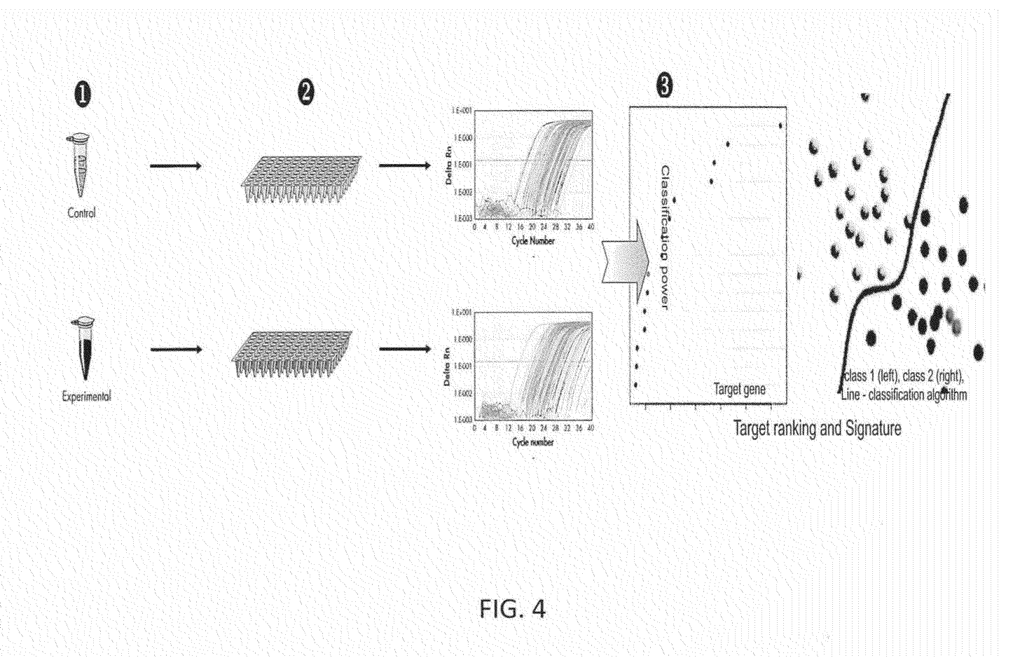

FIG. 4 shows a workflow from sample to biomarker signature panel using the disease-specific qPCR array system. Researcher's efforts: 1) Sample collection and processing, then 2) qPCR is performed to get C_{T} values, 3) Shows Data analysis portal:

A. Normalization of gene expression, with final normalization, gene panel selected based on expression stability of researcher's samples, to obtain ΔC_{T}.

B. Ranking of target genes for their classification power with RF ranking tool. Removal of unqualified targets (such as targets with no or low detection in both groups) for better assay stability.

C. Creation of a biomarker signature panel and classification algorithm using the RF model and cross validation.

### Development of Biomarker qPCR Array

In embodiments, methods of preparing a biomarker quantitative real-time polymerase chain reaction (qPCR) array are provided. Suitably, such methods comprise selecting one or more high-throughput feature expression data sets, normalising the feature expression data sets, analyzing the data sets by one or more mathematical models to yield final candidate features, and generating the biomarker qPCR array comprising the final candidate features.

As used herein, a “biomarker” refers to a measurable characteristic that provides information on presence and/or severity of a disease or compromised state in a patient; the relationship to a biological pathway; a pharmacodynamic relationship or output; a companion diagnostic; a particular species; or a quality of a biological sample. Examples of biomarkers include genes, proteins, peptides, antibodies, cells, gene products, enzymes, hormones, etc.

As used herein a “feature” refers to a genes, portions of genes or other genomic information. Suitably, a feature refers to a gene that is utilized to prepare an array as described herein.

In embodiments, the one or more high-throughput feature expression data sets (including microarray data sets, as well as other sequencing data sets including next generation sequencing platforms) are selected based on one or more of clinical utility (e,g. disease specific biomarkers), research interest (e.g., biological pathway-specific biomarkers), drug response (e.g., pharmacodynamic biomarkers or companion diagnostic biomarkers), species and quality.

In embodiments, the analyzing comprises analysis of the data sets with one or more mathematical models including but not limited to, Random forest (RF) modeling, support vector machine (SVM) modeling and nearest shrunken centroid (NSC) modeling. Additional models known, in the art can also be utilized in the methods described herein, including for example, various genetic algorithms, decision tress and Naïve Bayes modeling.

Methods of conducting such modeling are well known in the art, and described for example, RF models are described in Touw et al, “Data mining in the Life Sciences with Random Forest: a walk in the park or lost in the jungle?” Briefings in Bioinformatics, May 26, 2012, Kursa and Rudnicki, “The All Relevant feature Selection using Random Forest,” Cornell University Library, arXiv; 1106,5112, Jun. 25, 2011, Genuer et al., “Variable Selection using Random Forests,” Paper Submitted to Pattern Recognition Letters, Mar. 17, 2010, Ostroff et al., “Early Detection of Malignant Pleural Mesothelioma in Asbestos-Exposed Individuals with a Noninvasive Proteomics-Based Surveillance Tool,” PLOS ONE 7:e46091 (October 2012), Chen et al., “Development and Validation of a qRT-PCR Classifier for Lung Cancer Prognosis,” J. Thorac. Onocl. 6:1481-1487 (September 2011); NSC models are described in Klassen and Kim, “Nearest Shrunken Centroid as Feature Selection of Microarray Data, available at Http://www.researchgate.net/, Tibshirani et al., “Diagnosis of multiple cancer types by shrunken centroids of gene expression,” Proc. Natl. Acad. Sci. 99:6567-6572 (May 14, 2002); and SVM models are described in Yousef et al., “Classification and biomarker identification using gene network molecules and support vector machines,” BMC Bioinformatics 10:337 (2009), and Brank, J., “Feature Selection Using Linear Support Vector Machines,” Microsoft Research Technical Report, MSR-TR-2002-63 (Jun. 12, 2002) (the disclosure of each of which is incorporated by reference herein in their entireties, specifically for the disclosure of the models described herein and their implementation). In embodiments, the analysis comprises use of two, or more suitably, all three of these models on the data to generate the combined feature set and the final qPCR array.

Suitably, the analysing comprises combining discriminative features from one or more of the mathematical models based on a desired classification implied by the data sets. That is, depending on the desired analysis (i.e., clinical outcome, research interest, etc.), features that discriminate between one biomarker and another are selected. For example, genes that are present in a disease state are selected over genes that are not indicative of the disease state or other characteristic.

As described herein, the analysis can further comprise literature mining to yield the final candidate features. This allows for the addition of further information to clarify and define the desired candidate features.

Suitably, the methods further comprise selecting one or more control data sets for inclusion of control features in the biomarker qPCR. array. As described herein, it is the selection of these control features (i.e., features that do not demonstrate a change in a biomarker characteristic) that provides one of the unique features of the methods and arrays provided herein, so as to produce the most useful array information.

Also provided are qPCR arrays prepared by the methods described herein. In suitable embodiments, each defined location in an array corresponds to a biological target. For example, an array suitable comprises a feature selection (e.g., gene selection) such that each well of an array plate represents a target for analysis.

In embodiments, the qPCR arrays are designed for analysis of various biomarkers, including various nucleic acid molecules, for example, for analysis of messenger RNA (mRNA), for analysis of micro RNA (miRNA), for analysis of long non-coding RNA (IncRNA), etc as well as combinations thereof.

As described herein, in suitable embodiments the qPCR arrays comprise one or more, suitably two or more, three or more, four or more or five or more control features (i.e., genes) including, but not limited to: ACTB, B2M, GUSB, HPRT1, RPL13A, S100A6, TFRC, YWHAZ, CFL1, RPS13, TMED10, UBB, ATP5B, GAPDH, HMBS, HSPCB, RPLPO, SDHA, UBC, PP1A, FLOT2, TMB1M6, TBT1, MRPL19 and RPLP0. In suitable embodiments, the arrays comprise 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or all 25 of the control features described herein.

In further embodiments, additional control features (reference genes) can also be included in the qPCR arrays, including features from animals other than humans, including for example, mouse, rat, monkey, dog, etc. Such reference features can be selected by utilizing the various methods described herein applied to information from other animals.

Further exemplary reference features include, for example,

Mouse reference features:
Actb NM_{—}007393
B2m NM_{—}009735
Gapdh NM_{—}008084
Gusb NM_{—}010368
Hsp90ab1 NM_{—}008302

Rat reference features:
Actb NM_{—}031144
B2m NM_{—}012512
Hprt1 NM_{—}012383
Ldha NM_{—}017025
Rp1p1 NM_{—}001007604

Cow reference features:
ACTB NM_{—}173979
GAPDH NM_{—}001034034
HPRT1 NM_{—}001034035
TBP NM_{—}001075742
YWHAZ NM_{—}174814

Rhesus Macaque reference features;
ACTB NM_{—}001033084
B2M NM_{—}111047137
GAPDH XM_{—}001105471
LOC709186 XM_{—}001097691
RPL13A XM_{—}001115079

miRNA reference features:
SNORD61 MS00033705
SNORD68 MS00033712
SNORD72 MS00033719
SNORD95 MS00033726
SNORD96A MS00033733
RNU6-2 MS00033740

In still further embodiments, the methods described herein provide methods of assigning a single probability score to one or more biomarkers. Suitably, such methods comprise collecting a sample set. Suitably, such sample sets are nucleic acid solutions, but can also be cell or tissue samples, blood samples, saliva samples, urine samples or other biological fluid samples, and can further comprise various proteins or other biological materials.

Suitably, nucleic acid molecules are extracted from each sample of the sample set. Methods for carrying out $uch: extraction are well known in the art.

Each nucleic add molecule is then interrogated with the qPCR arrays as described herein. As used herein “interrogating” refers to applying the sample(s) to one or more locations (i.e., wells) of the array. The methods suitably comprise evaluating the discrimination power of one or more independent features. That is, the ability of one or more features (e.g., genes) of the array is evaluated to determine how well they discriminate between a characteristic of a biomarker (i.e., disease vs. non-disease state).

The methods further comprise generating a combined feature by analyzing the discrimination power of combinations of two or more independent features with one or more mathematical models. Methods for generating the combined feature, including the mathematical models utilised, are described herein and include for example. Random forest (RF) modeling, support vector machine (SVM) modeling and nearest shrunken centroid (NSC) modeling. Additional models known in the art can also be utilized in the methods described herein, including for example, various genetic algorithms, decision tress and Naïve Bayes modeling.

The methods then further comprise assigning a single probability score to the combined features. That is, a single value is assigned to the combined features that can be utilized to determine whether or not the level of a biomarker is indicative of the measured/desired outcome. The “cut-off” value for a biomarker—the probability score below or above which the presence of a biomarker is determinative—is suitably scalable, i.e., up or down as desired.

In exemplary embodiments, the interrogating comprises evaluating 2 to 40 independent features (i.e., genes) on a single array. As described herein, arrays are suitably 96 well plates, and thus the desired number of feature is suitably dependent upon the physical characteristics of the plates (number of wells in a row or column) and the ability to deposit the features (e.g., genes, etc.) on the plate. In suitable embodiments, the interrogating comprises evaluating 2 to 8 independent features, 8 to 16 independent features, 16 to 24 independent features, 24 to 32, independent features, 32 to 40 independent features, or 20 independent features, as well as values and ranges within these ranges.

As described herein, the focus of a disease specific biomarker can be selected based on market needs, customer request, collaboration, etc.

High-throughput gene expression data is selected based on the topic (from public database or from collaboration or a customer's own data).

Data is normalized and suitably an annotation file is downloaded.

The normalized data is used for feature selection. Mathematical models, RF, SVM and NSC, are used to rank genes based on their classification power and generate an independent list. All the lists are combined based on each gene's ranking in each list.

Literature mining is used to find well-accepted, publically recognized biomarker candidate genes (usually 25-50 genes) and added to the final list.

Reference genes are selected based on literature for their normalization power. Suitably, some clinical samples relevant to the topic are used to evaluate some of the potential reference gene expressions. geNorm gene expression stability analysis is used to pick suitable genes (in embodiments 9 reference genes are used in the final assay).

Gene target sequences are put into a primer design tool for assay design. Probe(s) are designed, and a qPCR primer pair is designed around each probe design. Suitably, an assay design set, including a pair of primer and a probe, are used.

The designed assay is evaluated with gnomic DNA for its performance (including sensitivity, specificity, efficiency, etc). Genes on the final candidate list that can get a qualified assay are kept for the final PCR array together with 9 reference gene assays and 3 controls assays.

Reference gene selection: References genes are selected based on literature search and/or real-samples based on the expression stability test. More stable expressed reference genes are used in the PCR array, and are further selected by the data analysis tool for best reference performance.

Assay performance controls include genomic DNA contamination controls, reverse transcription efficiency controls and qPCR performance controls to aid in identification of any low quality data.

### Use of Disease Specific Biomarber PCR Array

Related clinical samples (usually including two phenotypes, such as malignant and non-malignant) are collected based on final clinical needs.

The collected tissue total RNA is purified (sneb as QIAGEN RNeasy kit). RNA is further converted to cDNA with reverse transcription.

Quantitative real-time PCR is performed with the disease specific qPCR array in a qPCR instrument.

The gene expression data is exported from qRCR instrument with its attached software.

Raw data is uploaded to the data analysis tool. The data analysis tool evaluates the data quality with the control assays and reference genes assay. Low quality data are removed from analysis.

Reference genes are selected based on gene expression stability analysis. Target gene expression is normalized with the average of reference gepe expression. Normalized gene expression is input into a classification analysis model system (such as Random forest) to identify the best number of genes to be used for classification and which genes are to be used. An algorithm with model parameters is decided based on calculation and saved.

The resulting output is a gene list and related algorithm (or further validation.

The identified genes and calculation algorithm can be further developed into clinical biomarker by well designed clinical trial(s) to serve a diagnostic or prognostic purpose.

It will be readily apparent to one of ordinary skill in the relevant arts that other suitable modifications and adaptations to the methods and applications described herein can be made without departing from the scope of any of the embodiments. It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

### EXAMPLES

### Example 1

### Thyroid Malignancy qPCR Array

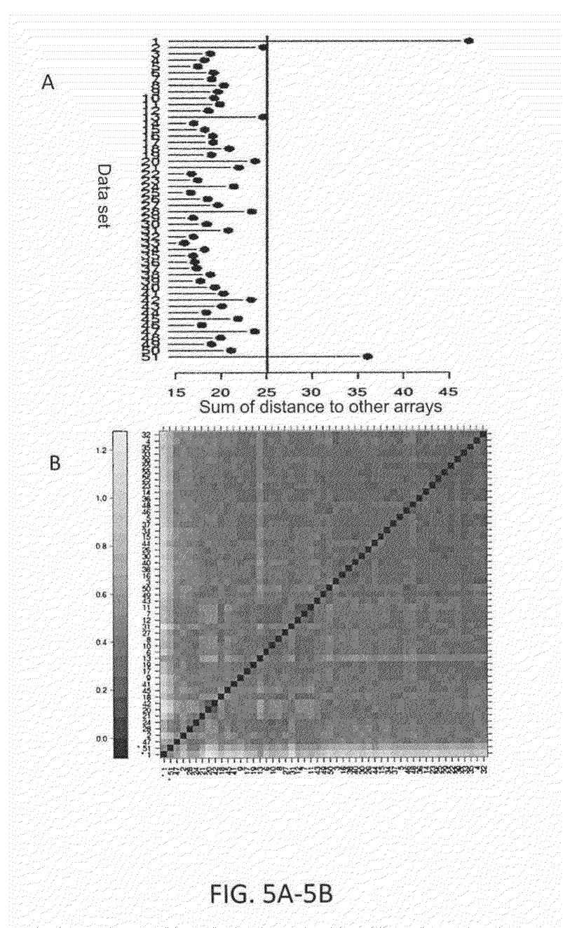

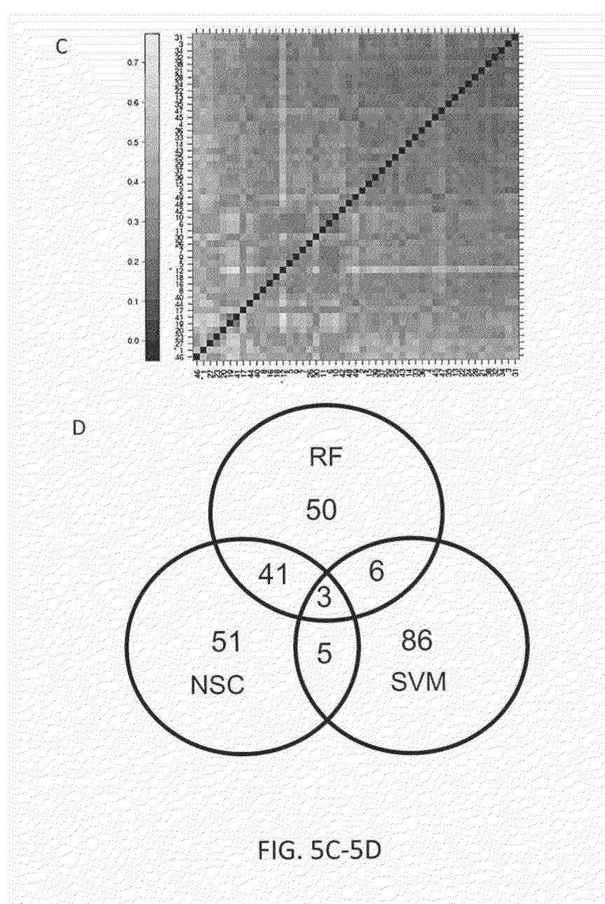

The published literature was searched, and published high-throughput screening (microarray) data from 51 benign and malignant thyroid samples were selected for study. Outlier samples were identified and are shown in FIG. 5A. Outlier samples were removed from the dataset because they impaired sample clustering as shown in FIG. 5B. Sample clustering improved with removal of the outliers as shown in FIG. 5C. Multiple mathematical models including RF, NSC and SVM were used for biomarker candidate selection, and genes selected based on the literature were added for better potential biomarker coverage. FIG. 5D shows the overlap of the top 100 genes across the three representative mathematical models. qPCR assays were then performed on the top-ranked targets and were optimized for their sensitivity, specificity and efficiency. Target assays meeting the QC standards were used for thyroid malignancy qPCR array. Normalization reference gene candidates were selected based on gene expression stability analysis with representative benign and malignant thyroid samples. Ultimately, 371 target assays, 10 normalization controls and 3 performance controls were used on a 384-well thyroid malignancy qPCR array.

Forty-nine pathology-assessed thyroid, nodule samples (fresh frozen, 23 malignant and 26 benign, Weill Medical College of Cornell University) were tested using the thyroid malignancy qPCR array. Normalization genes were selected based on gene expression stability and inter-group variation. The geometric mean of 5 selected normalization genes was used to normalize target gene expression. Normalized C_{T} values were analyzed using an RF classification model. The optimization algorithm identified a panel of 12 genes as a gene expression signature for thyroid malignancy, shown below in Table 1.

TABLE 1
Thyroid Malignancy Gene Expression Signature

| | | | | | |
|---|---|---|---|---|---|
| NPC2 | S100A11 | SDC4 | CD53 | MET | GCSH |
| CHI3L1 | TBP | RPL13A | RPS13 | HSP90AB1 | YWHAZ |

Twelve pathology-assessed thyroid nodule samples (RNA from fresh frozen tissue; 8 malignant and 4 benign) were evaluated using the identified thyroid malignancy gene expression signature and a companion classification algorithm. Malignant thyroid nodule samples were successfully distinguished from benign nodules samples with 92% accuracy and 100% specificity in this limited size, independent dataset, as shown in Table 2.

TABLE 2
Prediction Results

| | Accuracy | Sensitivity | Specificity | PPV | NPV |
|---|---|---|---|---|---|
| | (%) | (%) | (%) | (%) | (%) |
| Prediction | 91.7 | 87.5 | 100.0 | 100.0 | 80.0 |
| result | | | | | |

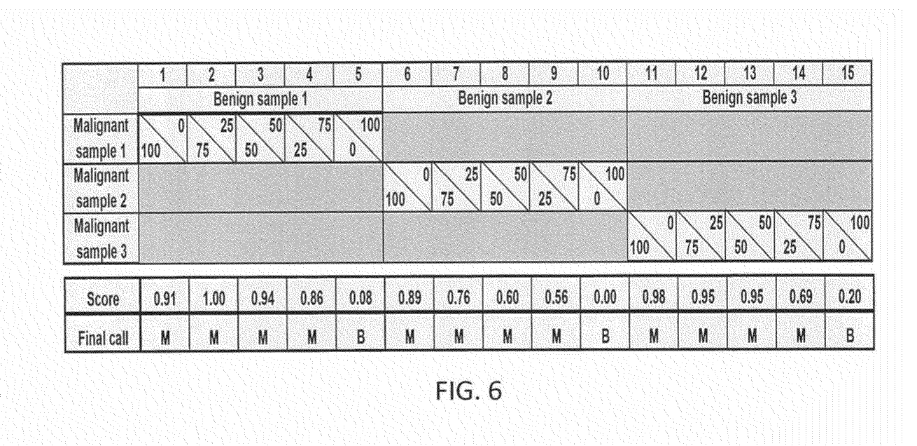

Three pairs of benign and malignant thyroid samples were mixed in different ratios and analyzed using the thyroid malignancy gene expression signature and companion classification algorithm. Analysis results provided a malignancy score for each sample and distinguished mixed samples containing as little as 25% malignant sample from pure benign samples with 100% accuracy, as shown in FIG. 6. Malignant-Score>0.5 (M), Benign-Score<0.5 (B).

### Example 2

### Development of Tuberculosis (TB) Infection Biomarker Introduction

Tuberculosis (TB) is a disease that is spread through the air from one person to another. It is caused by various strains of mycobacteria, usually Mycobacterium tuberculosis. More than 2 billion people are estimated to be infected with Mycobacterium tuberculosis in 2008 (6). In 2010, 8.8 million individuals became ill with TB and 1.4 million died [WHO report 2012].

There are two kinds of tests that are used to determine if a person has been infected with TB bacteria: the tuberculin skin test and T8 blood tests. The challenge is that skin test needs 48-72 h and blood test needs 24 h or more to get a result. In addition, a positive result doesn't mean active TB. For reduction in TB incidence, it is important to identity and treat the active TB patients rapidly.

qPCR has been widely used as a platform for biomarker assay development with its high sensitivity, wide dynamic range and fast turnaround time. This Example describes the development of a TB biomarker to discriminate active TB from both latent infection and uninfected status, as well as from other diseases.

### Results

1. Target Gene Selection

For identifying candidate biomarkers, the microarray study results from two cohorts in South Africa (SUN) and Gambia (MRC) were used. Those cohort studies used Agilent two-color microarray slides with PAXgene blood RNA samples. Microarray data was processed with the disclosed biomarker array feature selection system that utilizes bioinformatics models for selecting best candidates for further qPCR based studies. Literature mining was also used to get additional candidates for TB biomarker PCR array development. Top ranked genes were combined to generate a final target list.

2. Biomarker PCR Array Development

For generating a TB biomarker qPCR array, the Biomarker qPCR array primer set design system described herein was utilized to generate candidate primers. With genomic DNA based primer quality control for its sensitivity, specificity and efficiency, the biomarker qPCR array allowed for detection of all final targets properly with a qualified assay. Based on literature 9 reference genes were added as candidate references for further analysis, GDC, PPC and RTC control were also used for RT-qPCR performance control.

3. Biomarker qPCR Array Pilot Study

360 genes and a small number of samples wore used for selecting biomarker candidates and a developing a companion classification algorithm that successfully discriminated TB-infected and healthy individuals. 26 blood samples (17 from TB-infected patients and 9 from healthy donors) were analyzed at MPI with QIAGEN/MPI custom classifier qPCR array. After removing five failed samples (those with no C_{T} value for most of the assays), the qPCR data set from 21 samples (15 TB-infeeted and 6 healthy control samples) were analyzed by Random forest; a gene selection and classification algorithm. A Random forest method identified a panel of 16 genes as a putative gene expression signature for TB-infection, which led to the development of a trained classification algorithm for discriminating TB-infected and healthy individuals. An evaluation of the selected 16 genes and companion classification algorithm showed 90% average accuracy and an average area under ROC curve (AUC) of 0.99.

4. qPCR Array Data Tidying

The entire raw C_{T} dataset was evaluated sampie-by-sampie and gene-by-gene. Twenty-nine genes with a C_{T} value >35 or an “undetermined” C_{T} value in 10 or more samples were believed to have an extremely low level of expression and therefore not useful for classification. The distribution of these “absent calls” across all samples was first checked to insure no bias existed between the two sample groups. These genes were then removed from further analysis. Any remaining “undetermined” C_{T} values and C_{T} values >35 were converted to 35 for further analysis,

5. Reference Gene Analysis

The stability of the reference genes' expression was evaluated with the Bioconductor geNorm analysis R package called NormqPCR. The geometric mean (GEOMEAN) of the C_{T} values of the top five selected housekeeping: genes (RPLP0, EEF1A1, TBP, UBE2D2 and B2M) was calculated for each sample as its normalization factor, Delta C_{T} values (normalized relative gene expression levels) were calculated as the difference between each target genes' C_{T} value and the appropriate sample-specific normalization factor.

6. General Gene Expression Analysis

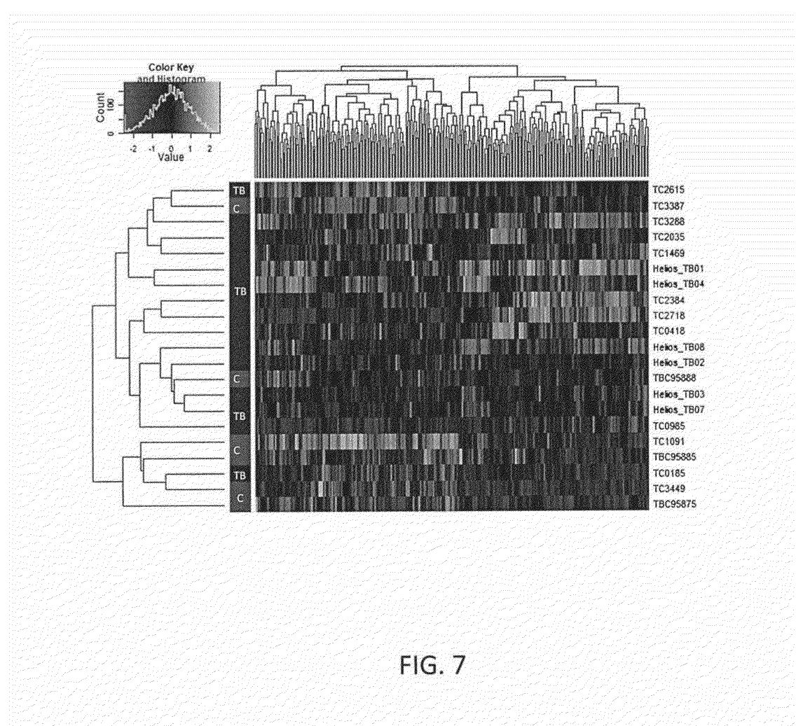

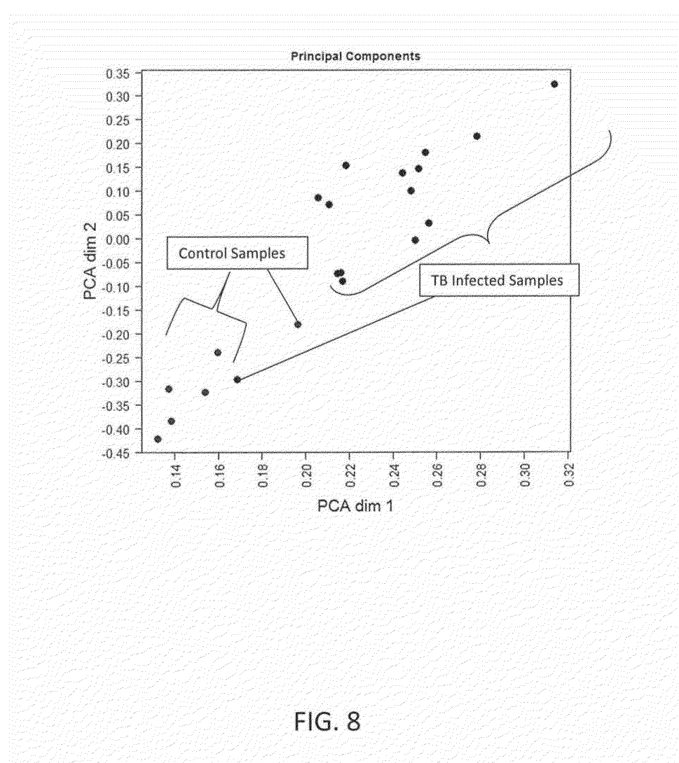

Two typical and standard methods of data analysis, unsupervised hierarchical clustering and principal component analysis (PCA), were first performed to check if the normalized gene expression levels would at least roughly classify the samples into the two expected groups. The results shown, in FIG. 7 and FIG. 8 indicate that although each method misclassifies two or three samples, the remaining samples classify well enough to apply more sophisticated methods and define a more limited and specific gene list that might classify the samples even better.

7. Gene Importance Ranking

The random forest R package known as “RandomForest” was used for analysing the qPCR data set and, in turn, determining gene importance for ranking, selecting potential bionwker genes, and developing a classification model.

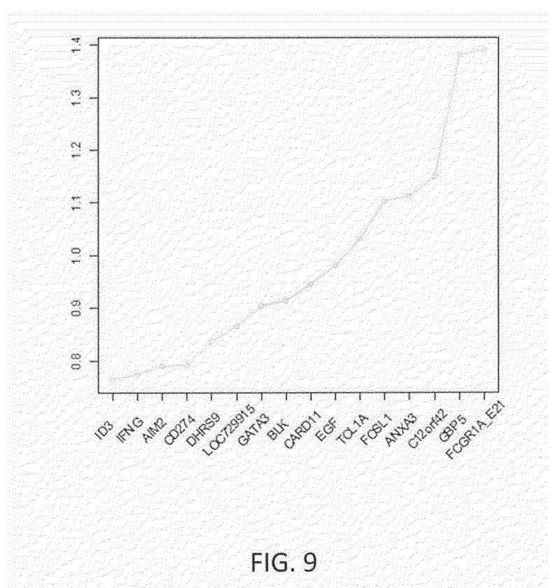

Random forest feature selection with 100 bootstraps was used to rank the genes based on their RF “permutation importance” in various classification models. To measure the importance of feature k (normalised gene expression) in RF trees, the values of this feature are randomly shuffled in the out-of-bag (OOB) samples. If Vk is the difference in classification accuracy between the intact OOB samples and the OOB samples with a particular feature permutated, then the RF “permutation importance” for feature k is defined as the average of Vk over ail trees in the forest. FIG. 9 then plots the median of 100 “permutation importance” values for the top ranked genes based on this analysis.

8. Potential Biomarker Selection and Classification Algorithm Development

Classification analysis was performed with Random forest models using different numbers and sets of genes. Parameters measuring performance were calculated for all models, and the median values of those parameters were determined for each set of models including the same number of genes. The number of genes in a model did not have dramatic effects on most of the measures of classification performance determined, accuracy for example (Table 3). This phenomenon tends to be caused by one of two reasons: 1) a limited number of samples or 2) a set of top-ranked genes that already classifies the samples very well while additional genes do not significantly add any more value (consistent with the results of FIG. 9). Based on the shortest list of genes required to maximize the AUC, the top ranked 16 genes were chosen as the putative signature panel (Table 4).

TABLE 3
The number of genes used to build classification models
did not dramatically affect the models' classification
powers. The table displays the median value for different
measurements of classification performance across several
models using different numbers of genes, from 2 to 330.

| Gene | | | | | | | |
|---|---|---|---|---|---|---|---|
| Number | TPR | FPR | SPC | PPV | NPV | ACC | AUC |
| 330 | 0.800 | 0.167 | 0.833 | 0.923 | 0.625 | 0.810 | 0.850 |
| 40 | 0.800 | 0.167 | 0.833 | 0.923 | 0.625 | 0.810 | 0.900 |
| 32 | 0.867 | 0.167 | 0.833 | 0.929 | 0.714 | 0.857 | 0.911 |
| 24 | 0.800 | 0.167 | 0.833 | 0.923 | 0.625 | 0.810 | 0.894 |
| 16 | 0.867 | 0.167 | 0.833 | 0.929 | 0.714 | 0.857 | 0.911 |
| 8 | 0.800 | 0.167 | 0.833 | 0.923 | 0.625 | 0.810 | 0.872 |
| 2 | 0.867 | 0.167 | 0.833 | 0.929 | 0.714 | 0.857 | 0.861 |

TPR: True Positive Rate
FPR: False Positive Rate
SPC: Specificity
PPV: Positive Predictive Value
NPV: Negative Predictive Value
ACC: Accuracy
AUC: Area Under ROC Curve

TABLE 4
Signature Gene Panel. The 16 top-ranked genes
are listed in decreasing order of importance (final
rank) along with their gene description.

| Find | | |
|---|---|---|
| Rank | Symbol | Description |
| 1 | FCGR1A | Fc fragment of IgG, high affinity 1a, receptor |
| | | (CD64) |
| 2 | GBP5 | Guanylate binding protein 5 |
| 3 | FOSL1 | FOS-like antigen 1 |
| 4 | ANXA3 | Annexin A3 |
| 5 | C12orf42 | Chromosome 12 open reading frame 42 |
| 6 | CARD11 | Caspase recruitment domain family, member 11 |
| 7 | EGF | Epidermal growth factor |
| 8 | TCL1A | T-cell leukemia/lymphoma 1A |
| 9 | BLK | B lymphoid tyrosine kinase |
| 10 | GATA3 | GATA binding protein 3 |
| 11 | DHR89 | Dehydrogenase/reductase (SDR family) member 9 |
| 12 | LOC729915 | Putative POM121-like protein 1 |
| 13 | CD274 | CD274 molecule |
| 14 | AIM2 | Absent in melanoma 2 |
| 15 | IFNG | Interferon, gamma |
| 16 | ID3 | Inhibitor of DNA binding 3, dominant negative |
| | | helix-loop-helix protein |

6. Gene Signature Panel Evaluation

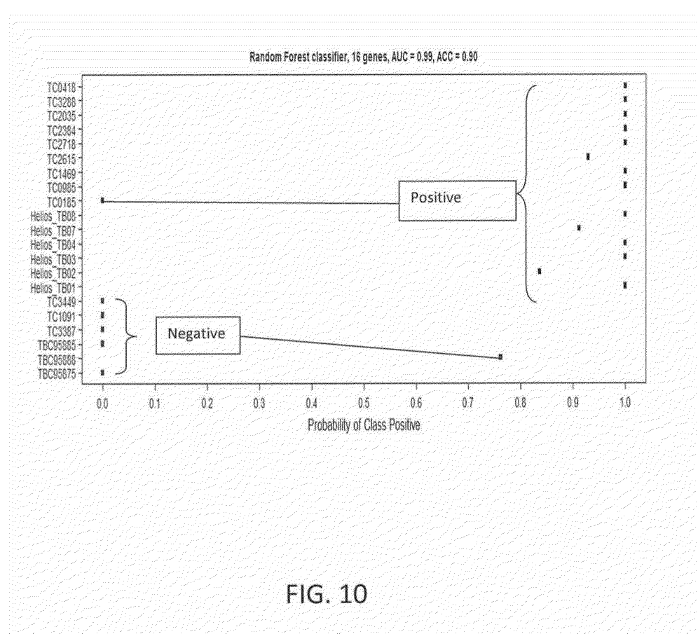

The performance of the 16-gene signature and the companion classification model was evaluated once again using the Random forest algorithm (Table 5). The evaluation process involved resampling of the initial dataset. Each resampling used a randomly selected set of healthy control samples and an equal number of TB-infected samples as its training set, and then classified the remaining samples using the 16-gene model. The classification decision for each test set was recorded. The probability that each sample was classified as TB-infected was finally calculated (FIG 10).

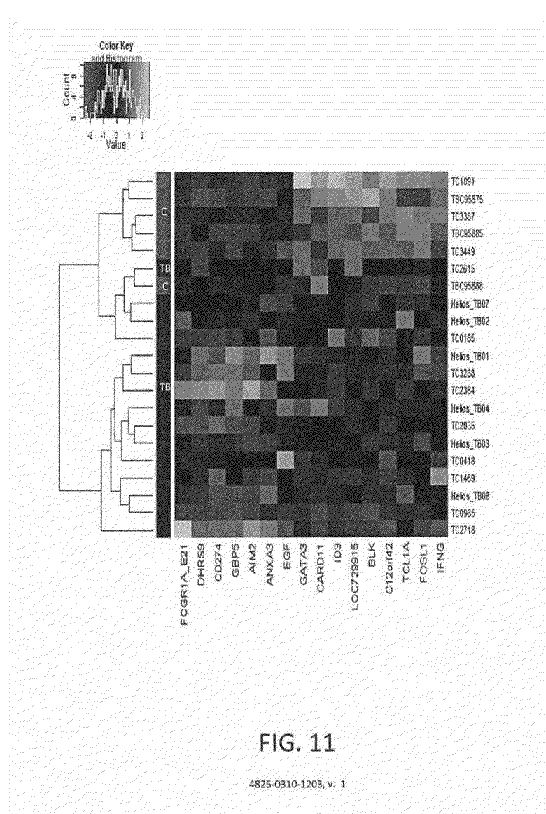

The same two samples misclassified by the original PCA (FIG. 8) continue to be misclassified in this model (TB-infected TC0185 and healthy control TBC95888), while the model also does not return a 100% probability for three TB-infected samples (TC2615, Helios_TB07, and Helios_TB02) unlike the other TB-infected samples. A new unsupervised hierarchical clustering analysis, using the final 16 gene panel (FIG. 11) better segregates the samples than the original cluster analysis (FIG. 7). However, it also seems to segregate the two misclassified samples and the three samples with “marginal calls” into a sub-group or third group of samples.

The continued misclassification of some samples during signature evaluation may again be due to the small number of samples used, causing under-representation during re-sampling. The planned study using a larger sample size in. both groups should help resolve these issues and questions.

TABLE 5
Final 16 gene signature evaluation result

| Gene | | | | | | | |
|---|---|---|---|---|---|---|---|
| Number | TPR | FPR | SPC | PPV | NPV | ACC | AUC |
| 16 | 0.933 | 0.167 | 0.833 | 0.933 | 0.833 | 0.905 | 0.989 |

TPR: True Positive Rate
FPR: False Positive Rate
SPC: Specificity
PPV: Positive Predictive Value
NPV: Negative Predictive Value
ACC: Accuracy
AUC: Area Under ROC Curve

### DISCUSSION & CONCLUSIONS

The highly ranked genes found here correlated well with previous studies. For example, the top ranked gene in this study, FCGR1A, was also found to be one of the strongest differentially expressed genes in tne Gambian cohort (1), FCGR1A and GBP5 (the second ranked gene here) were also identified as 2 out of the 4 most differentially expressed genes between active TB and normal individuals in Thai study (2).

### REFERENCES

1. Maertzdorf J, Ota M, Repsilber D, Mollenkopf H J, Weiner J, Hill P C, Kaufmann S H, Functional correlations of pathogenesis-driven gene expression signatures in tuberculosis. PLoS One, 2011;6(10):e26938, Epub 2011 Oct. 28, PubMed PMID: 22046420; PubMed Central PMCID: PMC3203931.

2. N. Satproedprai1, S. Mahasirimongkol1, W. Inunchot1, C. Somboonyosdech1, S. Kumperasart1, S. Wattanapokayakit1, K. Higuchi2, H. Yanai3, N. Harada2, N. Wichukchinda1 Validation of blood transcriptional signatures for tuberculosis infection in Thai population, 15th International Congress on Infectious Diseases. Bangkok, 2012

3. Ioan-Facsinay, A., S. J. de Kimpe, S. M. Hellwig, P. L. van Lent, P, M. Hofhuis, H. H. van Ojik, C. Sedlik S. A, da Silveira, J. Gerber, Y. P. de Jong, R, Roozendaal, L. A. Aarden, W. B, van den Berg, T. Saito, D. Mosser, S. Amigorena, S. Izui, G. -J. B. van Ommen, M. van Vugt, J. G. van de Winkel, and J. S. Verbeek. 2002. FegRI (CD64) contributes substantially to severity of arthritis, hypersensitivity responses, and protection from bacterial infection. Immunity 16;391-402.

4. Ito Y, Shibata-Watanabe Y, Ushijima Y, Kawada J, Nishiyama Y, Kojima S, imura. H. Oligonucleotide microarray analysis of gene expression profiles followed by real-time reverse-transcriptase polymerase chain reaction assay in chronic active Epstein-Barr virus infection, J Infect Dis. 2008 Mar. 1;197(5):663-6.

5. Shenoy A R, Wellington D A, Kumar P, Kassa H, Booth C J, Cresswell P, MacMicking J D, GBP5 promotes NLRP3 inflammasome assembly and immunity in mammals. Science. 2101 Apr 27;336(6080);481-5.

6. Lönnroth K, Raviglione M. Global epidemiology of tuberculosis: prospects for control. Semin Respir Crit Care Med. 2008 Oct;29(5):481-91.

All publications, patents and patent applications mentioned in this specification are herein incorporated by reference to the same extent as if each individual publication, patent or patent application was specifically and individually indicated to be incorporated by reference.

What is claimed is:

1. A method of preparing a biomarker quantitative real-time polymerase, chain reaction (qPCR) array, comprising:
a. selecting one or more high-throughput feature expression data sets;
b. normalizing the feature expression data sets;
c. analyzing the data sets by one or more mathematical models to yield final candidate features; and
d. generating the biomarker qPCR array comprising the final candidate features.

2. The method of claim 1, wherein the one or more high-throughput feature expression data sets are selected based on one or more of clinical utility, research interest, drug response, species and quality.

3. The method of claim 1, wherein the analyzing comprises analysis with one or more mathematical models selected from the group consisting of Random forest (RF) modeling, support vector machine (SVM) modeling and nearest shrunken centrold (NSC) modeling.

4. The method of claim 3, wherein the analyzing comprises combining discriminative features from one or more of the mathematical models based on a desired classification implied by the data sets.

5. The method of claim 1, wherein the analyzing further comprises literature mining to yield the final candidate features.

6. The method of claim 1, further comprising selecting one or more control data sets for inclusion of control features in the biomarker qPCR array.

7. A qPCR array prepared by the method of claim 1.

8. The qPCR array of claim 7, wherein each defined location in the array corresponds to a biological target.

9. The qPCR array of claim 8, wherein the qPCR array is for analysis of any one of messenger RNA (mRNA), micro RNA (miRNA), long non-coding RNA (lncRNA) and combinations thereof.

10. An qPCR array of claim 8, comprising five or more control features selected from the group consisting of: ACTB, B2M, GUSB, HPRT1, RPL13A, S100A6, TFRC, YWHAZ, CFL1, RPS13, TMED10, UBB, ATP5B, GAPDH, HMBS, HSPCB, RPLPO, SDHA, UBC, PPIA, FLOT2, TMB1M6, TBT1, HMBS, HSPCB, RPLPO, SDHA, UBC, PPIA, FLOT2, tMB1M6, TBT1, MRPL19 and RPLP0.

11. A method of assigning a single probability score to one or more biomarkers comprising;
a. collecting a sample set;
b. extracting nucleic acid molecules from each sample of the sample set;
c. interrogating each nucleic acid molecule with the qPCR array of claim 7 and evaluating the discrimination power of one or more independent features;
d. generating a combined feature by analyzing the discrimination power of combinations of two or more independent features with one or more mathematical models; and
e. assigning a single probability score to the combined features.

12. The method of claim 11, wherein the interrogating comprises evaluating 2 to 40 independent features.

13. The method of claim 12, wherein the interrogating comprises evaluating 2 to 8 independent features.

14. The method of claim 12, wherein the interrogating comprises evaluating 8 to 16 independent features.

15. The method of claim 12, wherein the interrogating comprises evaluating 16 to 24 independent features.

16. The method of claim 12, wherein the interrogating comprises evaluating 24 to 32 independent features.

17. The method of claim 12, wherein the interrogating comprises evaluating 32 to 40 independent features.

18. The method of claim 12, wherein the interrogating comprises evaluating 20 independent features.
